(19) United States
(12) Patent Application Publication
(10) Pub. No.: US 2021/0386864 A1
(43) Pub. Date: Dec. 16, 2021

(54) CYTOSTATIC CONJUGATES WITH INTEGRIN LIGANDS

(71) Applicant: Bayer Pharma Aktiengesellschaft, Berlin (DE)

(72) Inventors: Hans-Georg LERCHEN, Leverkusen (DE); Beatrix STELTE-LUDWIG, Wülfrath (DE); Charlotte Christine KOPITZ, Falkensee (DE); Jörg KELDENICH, Berlin (DE)

(21) Appl. No.: 17/290,911

(22) PCT Filed: Oct. 30, 2019
(86) PCT No.: PCT/EP2019/079601

§ 371 Date: May 3, 2021

(30) Foreign Application Priority Data
Nov. 5, 2018 (EP) 18204423.0

(51) Int. Cl. A61K 47/65; A61K 47/60; A61K 47/54

(52) U.S. Cl. CPC A61K 47/65; A61K 47/545; A61K 47/60

(57) ABSTRACT

The present invention relates to novel pharmaceutical compounds comprising of an αᵥβ₃ integrin antagonist, a linking unit comprising of L-Val-L-Pro-L-Asp cleavable by elastase, a polyethyleneglycol (PEG) spacer and a cytotoxic element, to processes for preparation thereof, to the use thereof for treating, preventing or managing diseases and conditions including hyperproliferative disorders such as cancer in humans and other mammals.

The present invention relates to novel pharmaceutical compounds comprising of an αᵥβ₃ integrin antagonist, a linking unit comprising of L-Val-L-Pro-L-Asp cleavable by elastase, a polyethyleneglycol (PEG) spacer and a cytotoxic element, to processes for preparation thereof, to the use thereof for treating, preventing or managing diseases and conditions including hyperproliverative disorders such as cancer in humans and other mammals.

Chemotherapy in cancer is accompanied by usually serious side effects which are to be attributed to the toxic action of chemotherapeutics on proliferating cells of other tissue types than tumor tissue. For many years, scientists have occupied themselves with the problem of improving the selectivity of active compounds employed. A frequently followed approach is the synthesis of prodrugs which are released more or less selectively in the target tissue, for example, by change of the pH (DE-A 42 29 903), by enzymes (e.g. glucuronidases; EP-A 511 917 and 595 133) or by antibody-enzyme conjugates (WO 88/07378; U.S. Pat. No. 4,975,278; EP-A 595 133). A problem in these approaches is, inter alia, the lack of stability of the conjugates in other tissues and organs, and in particular the ubiquitous active compound distribution which follows the extracellular release of active compound in the tumor tissue.

20(S)-Camptothecin is a pentacyclic alkaloid which was isolated in 1966 by Wall et al. (J. Am. Chem. Soc. 88, 3888 (1966)). It has a high active antitumor potential in numerous in-vitro and in-vivo tests. Unfortunately, however, the realization of the promising potential in the clinical investigation phase failed because of toxicity and solubility problems.

By opening of the E ring lactone and formation of the sodium salt, a water-soluble compound was obtained which is in a pH-dependent equilibrium with the ring-closed form. Here too, clinical studies have not led to success as yet.

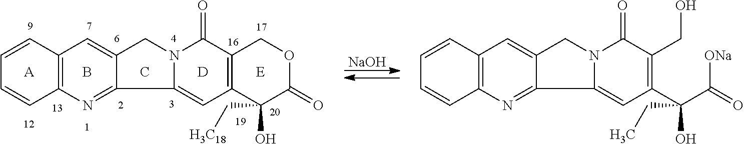

About 20 years later, it was found that the biological activity is to be attributed to enzyme inhibition of topoisomerase I. Since then, the research activities have again been increased in order to find a camptothecin derivative which is more soluble and more tolerable and which is active in-vivo.

For improvement of the water solubility, salts of A-ring- and B-ring-modified camptothecin derivatives and of 20-O-acyl derivatives with ionizable groups have been described (U.S. Pat. No. 4,943,579). The latter prodrug concept was later also transferred to modified camptothecin derivatives (WO 96/02546). The described 20-O-acyl prodrugs, however, have a very short half-life in vivo and are very rapidly cleaved to give the parent structure.

Integrins are heterodimeric transmembrane proteins found on the surface of cells, which play an important part in the adhesion of the cells to an extracellular matrix. They recognize extracellular glycoproteins such as fibronectin or vitronectin on the extracellular matrix via the RGD sequence occurring in these proteins (RGD is the single-letter code for the amino acid sequence arginine-glycine-aspartate).

In general, integrins such as, for example, the vitronectin receptor, which is also called the αᵥβ₃ receptor, or alternatively the αᵥβ₅ receptor or the GpIIb/IIIa receptor play an important part in biological processes such as cell migration, angiogenesis and cell-matrix adhesion and thus for diseases in which these processes are crucial steps. Cancer, osteoporosis, arteriosclerosis, restenosis and ophthalmia may be mentioned by way of example.

The αᵥβ₃ receptor occurs, for example, in large amounts on growing endothelial cells and makes possible their adhesion to an extracellular matrix. The αᵥβ₃ receptor thus plays an important part in angiogenesis, i.e. the formation of new blood vessels, which is a crucial prerequisite for tumor growth and metastasis formation in carcinomatous disorders.

It was possible to show that the blockade of the above-mentioned receptors is an important starting point for the treatment of disorders of this type. If the adhesion of growing endothelial cells to an extracellular matrix is suppressed by blocking their corresponding integrin receptors, for example, by a cyclic peptide or a monoclonal antibody, angiogenesis does not occur, which leads to a stoppage or regression of tumor growth (cf., for example, Brooks et al. in Cell 79, 1157-1164 (1994)).

WO 98/10795 describes conjugates in which a molecule targeting tumors is linked to a functional unit such as, for example, a cytostatic or a detectable label such as, for example, a radioactive nuclide. Inter alia, integrin antagonists such as, for example, peptides having the RGD sequence described above are described as molecules targeting tumors or tumor stroma. Doxorubicin is described as an example of a cytostatic which is linked to a molecule of this type addressing tumors.

In the case of the compounds of WO 98/10795, the linkage is carried out such that the molecule addressing a tumor and the functional unit are directly bonded to one another with retention of their respective properties (cf., for example, p. 56, 1. 17, to p. 58, 1. 10, and Ex. 6). This has the result that these compounds are indeed selectively concentrated in the immediate vicinity of tumor cells by binding of the entity addressing a tumor (in the case of a radical having αᵥβ₃ integrin-antagonistic action by binding to the αᵥβ₃ integrin receptor which, in particular, is expressed on endothelial cells newly formed by angiogenesis), but on account of the direct combination the functional unit such as, for example, a cytostatic cannot be released into the intracellular space of the tumor tissue.

Fundamentally, the conjugate which on the one hand is selectively concentrated in tumor tissue by the effect of a part addressing αᵥβ₃ or αᵥβ₅ integrin receptors found in the conjugate, but on the other hand comprises a cytostatic which can be released from the conjugate, should have an increased toxophoric effect on tumor tissue due to the possibility of the more direct action of the cytostatic on the tumor cells compared with the conjugates described in WO 98/10795. In particular, such a toxophoric effect and tumor selectivity should even be higher, if the release of the cytostatic takes place in the immediate vicinity of the tumor tissue or even in the tumor cells.

In WO 00/69472 enzyme-activated anti-tumor prodrug compounds are disclosed which can be specifically cleaved by collagenase (IV) and elastase. With respect to linking units cleavable by elastase this application describes that the specific tetrapeptide sequences Ala-Ala-Pro-Val and Ala-Ala-Pro-Nva are suitable therefore. Furthermore, in this reference, no conjugates which comprise a moiety addressing αᵥβ₃ integrin receptors and a cytostatic are mentioned.

Y. Liu et al. (Mol. Pharmaceutics 2012, 9, 168) describe conjugates of Auristatins linked to an αᵥβ₃ integrin targeting moiety via an legumain-cleavable linker.

In EP 1 238 678 conjugates with cytotoxic agents are disclosed which target αᵥβ₃ integrins and have peptide linkers which can be specifically cleaved by elastase. With respect to linking units cleavable by elastase this application describes peptide sequences comprising Pro-Val and Pro-Leu which are suitable therefore. As toxophore moieties camptothecin and a quinolone carboxylic acid are exemplified.

Particular challenges of such conjugates include
sufficient solubility enabling intravenous administration in appropriate vehicles,
high tumor penetration of intact conjugates,
high stability in plasma to avoid systemic de-conjugation,
efficient binding to the targeted receptors in tumor microenvironment,
efficient cleavage by enyzymes present in tumor microenvironment,
high stability and cellular permeability of cleaved toxophore moieties to enhance tumor cell uptake versus re-distribution.

It is therefore one objective of the present invention to develop conjugates which comprise a moiety addressing αᵥβ₃ integrin receptors and a cytostatic which can be released from the conjugate preferably in tumor microenvironment, where the moiety in the conjugate addressing αᵥβ₃ integrin receptors retains its ability to bind to the αᵥβ₃ integrin receptor and therefore provides tissue selectivity to such compounds. In addition, cleavability of the conjugates and drug release should be mediated by enzymes present and active in the tumor environment such as neutrophil elastase. Finally, the profile of the toxophore should match an extracellular cleavage and release mechanism in a way, that it should be highly permeable into tumor cells and tissues and not being a substrate of drug transporters.

The present invention relates to pharmaceutical compounds which are conjugates comprising an αᵥβ₃ integrin antagonist, linking units which can be selectively cleaved by elastase, a polyethyleneglycol (PEG) spacer and a cytotoxic element (toxophore). The conjugates have a tumor-specific action as a result of linkage to αᵥβ₃ integrin antagonists via preferred linking units which can be selectively cleaved by elastase, i.e. by an enzyme which can especially be found in tumor stroma. The preferred linking units provide sufficient stability of the conjugate of cytostatic and αᵥβ₃ integrin antagonist in biological media, e.g. culture medium or serum and, at the same time, the desired intracellular action within tumor tissue as a result of its specific enzymatic or hydrolytic cleavability with release of the cytostatic.

In particular, the compounds of the present invention show favorable features:
Improved stability of the conjugates after replacement of thio urea by urea linkage
Employment of 7-Ethyl camptothecin as a particularly suitable toxophore moiety
   Beneficial impact e.g. on lactone ring stability (Drugs Fut 2002, 27(9), 869)
   High cellular permeability and low efflux (as compared e.g. to SN38)
Modified spacer with beneficial impact on solubility, integrin-binding affinity, elastase cleavability
Tumor accumulation of toxophore after conjugate administration versus direct administration.
Excellent therapeutic efficacy in various tumor models.

Towards this goal, 7-Ethyl camptothecin is particularly preferred as the toxophore moiety in above mentioned conjugates.

The present invention provides compounds of the formula (I)

CT-LI-SP-IA (I)

in which
CT is a mono valent radical from the group of a cytotoxic radical, a radical of a cytostatic and a radical of a cytostatic derivative, which can each additionally carry hydroxyl, carboxyl or amino group
LI is a bivalent peptide radical of the formula: -L-Val-L-Pro-L-Asp-
SP is a group of the formula: —C═O—(CH₂)ₓ—O—(CH₂—CH₂—O)_{y}—CH₂—CH₂—NH—C═O— with x=1-5 and y=0-15
IA is a monovalent radical addressing an αᵥβ₃ integrin receptor
   and the salts, solvates and solvates of the salts thereof.

The bivalent peptide radial LI can be bound to CT or SP via its N-terminal or C-terminal position. Preferably LI is bound to CT via its C-terminal position and to SP via its N-terminal position.

The present invention further provides compounds of the general formula (Ta)

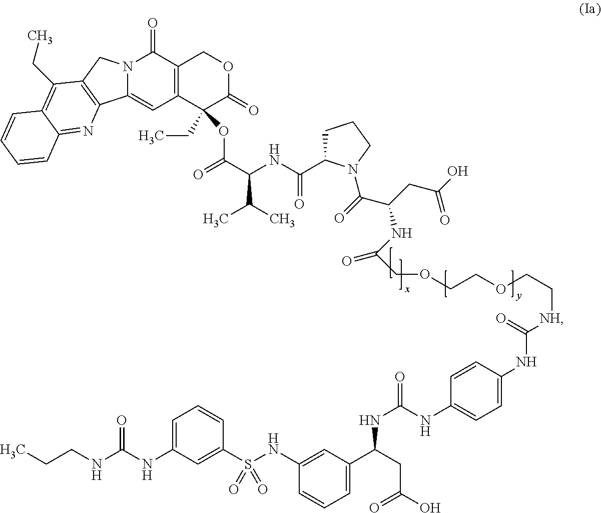

in which x is 1-5 and y=0-15,
and the salts, solvates and solvates of the salts thereof.

Preference is given to a compound of formula (I) or (Ta) in which x is 1-4, more preferred is a compound of formula (Ta) in which x is 1-2, most preferred is a compound of formula (Ta) in which x is 2.

Preference is given to a compound of formula (I) or (Ta) in which y is 0-10, more preferred is a compound of formula (Ta) in which y is 0-5, most preferred is a compound of formula (Ta) in which y is 2.

Preference is given to a compound of formula II:

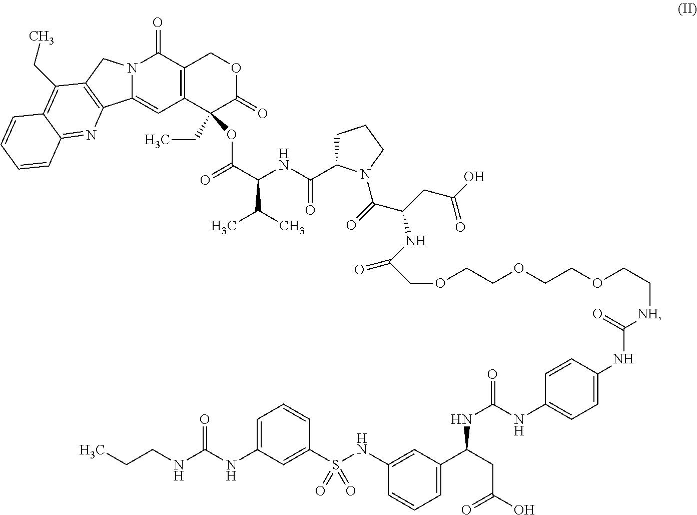 [SMILES: CCCNC(=O)Nc1cccc(S(=O)(=O)Nc2cccc([C@H](CC(=O)O)NC(=O)Nc3ccc(NC(=O)NCCOCCOCCOCC(=O)N[C@@H](CC(=O)O)C(=O)N4CCC[C@H]4C(=O)N[C@H](C(=O)O[C@]4(CC)C(=O)OCc5c4cc4n(c5=O)Cc5c-4nc4ccccc4c5CC)C(C)C)cc3)c2)c1]

and the salts, solvates and solvates of the salts thereof.

Preferred salts in the context of the present invention are physiologically acceptable salts of the inventive compounds. Also encompassed are salts which are not themselves suitable for pharmaceutical applications but can be used, for example, for the isolation, purification or storage of the inventive compounds.

Physiologically acceptable salts of the inventive compounds especially include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, benzenesulphonic acid, toluenesulphonic acid, naphthalenedisulphonic acid, formic acid, acetic acid, trifluoroacetic acid, propionic acid, succinic acid, fumnaric acid, maleic acid, lactic acid, tartaric acid, malic acid, citric acid, gluconic acid, benzoic acid and embonic acid.

In addition, physiologically acceptable salts of the inventive compounds also include salts derived from conventional bases, by way of example and with preference alkali metal salts (e.g. sodium and potassium salts), alkaline earth metal salts (e.g. calcium and magnesium salts), zinc salts and ammonium salts derived from ammonia or organic amines having 1 to 20 carbon atoms, by way of example and with preference ethylamine, diethylamine, triethylamine, N,N-ethyldiisopropylamine, monoethanolamine, diethanolamine, triethanolamine, dimethylaminoethanol, diethylaminoethanol, tris(hydroxymethyl)aminomethane, choline, benzalkonium, procaine, dibenzylamine, dicyclohexylamine, N-methylmorpholine, N-methylpiperidine, arginine, lysine and 1,2-ethylenediamine.

Preferred salt is the disodium salt of the compound of formula (II).

Solvates in the context of the invention are described as those forms of the inventive compounds which form a complex in the solid or liquid state by coordination with solvent molecules. Hydrates are a specific form of the solvates in which the coordination is with water. Solvates preferred in the context of the present invention are hydrates.

The present invention also encompasses all suitable isotopic variants of the inventive compounds. An isotopic variant of an inventive compound is understood here to mean a compound in which at least one atom within the inventive compound has been exchanged for another atom of the same atomic number, but with a different atomic mass than the atomic mass which usually or predominantly occurs in nature. Examples of isotopes which can be incorporated into an inventive compound are those of hydrogen, carbon, nitrogen, oxygen, phosphorus, sulphur, fluorine, chlorine, bromine and iodine, such as 2H (deuterium), ³H (tritium), ¹³C, ¹⁴C, 15N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I. Particular isotopic variants of an inventive compound, especially those in which one or more radioactive isotopes have been incorporated, may be beneficial, for example, for the examination of the mechanism of action or of the active ingredient distribution in the body; due to comparatively easy preparability and detectability, particularly compounds labelled with ³H, ¹⁴C and/or ¹⁸F isotopes are suitable for the purpose. In addition, the incorporation of isotopes, for example of deuterium, can lead to particular therapeutic benefits as a consequence of greater metabolic stability of the compound, for example an extension of the half-life in the body or a reduction in the active dose required; such modifications of the inventive compounds may therefore possibly also constitute a preferred embodiment of the present invention. Isotopic variants of the inventive compounds can be prepared by commonly used processes known to those skilled in the art, for example by the methods described further down and the procedures described in the working examples, by using corresponding isotopic modifications of the respective reagents and/or starting compounds.

The synthesis of the conjugates of the current invention (e.g. example 1) is outlined in the schemes below

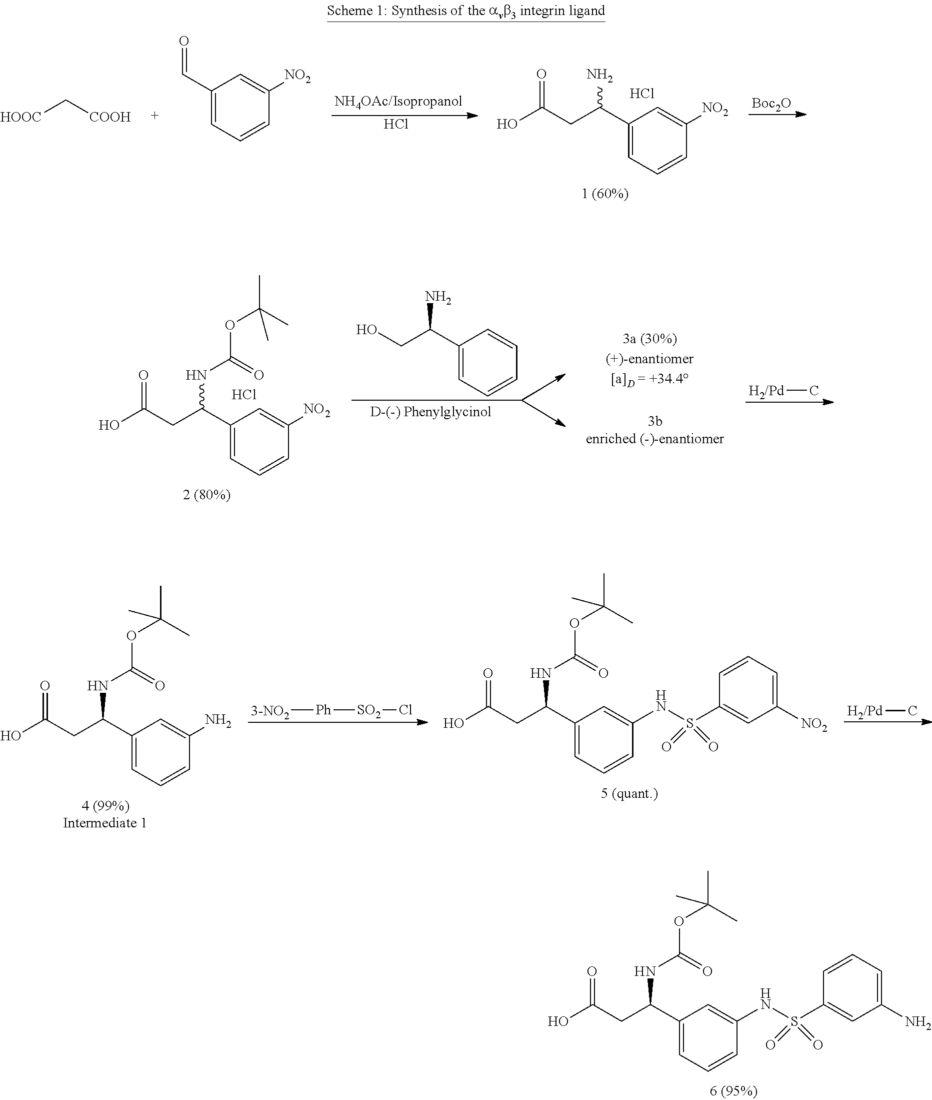

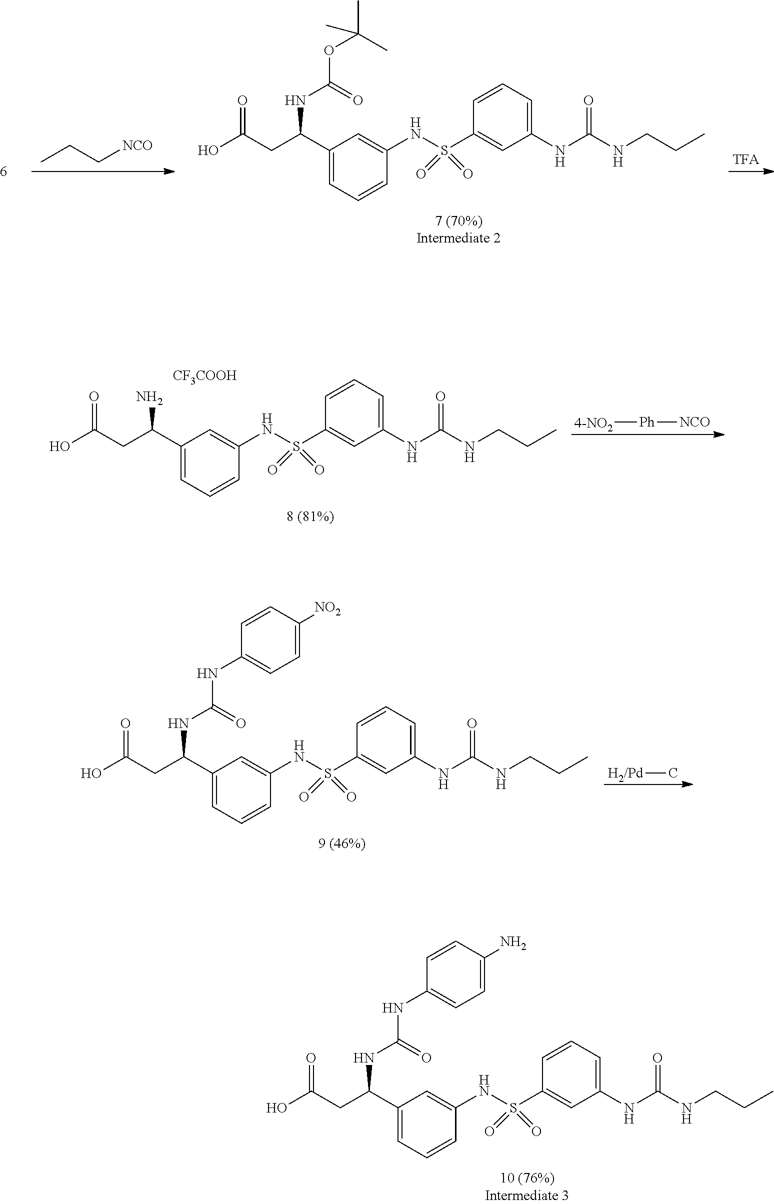

Separation of enantiomers can also be accomplished on different steps via chromatography using chiral columns.

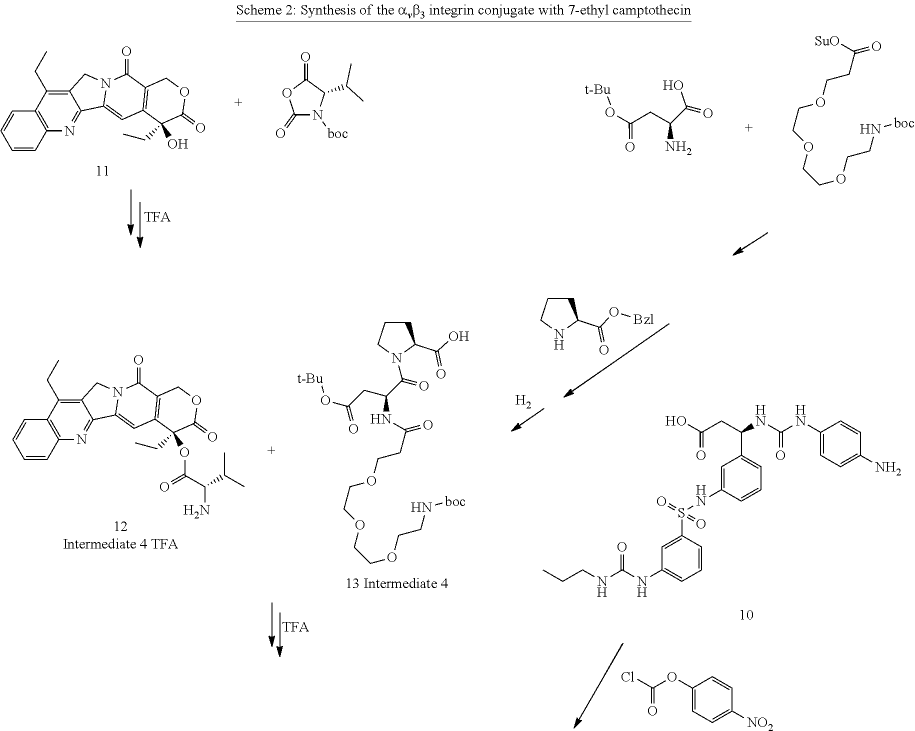

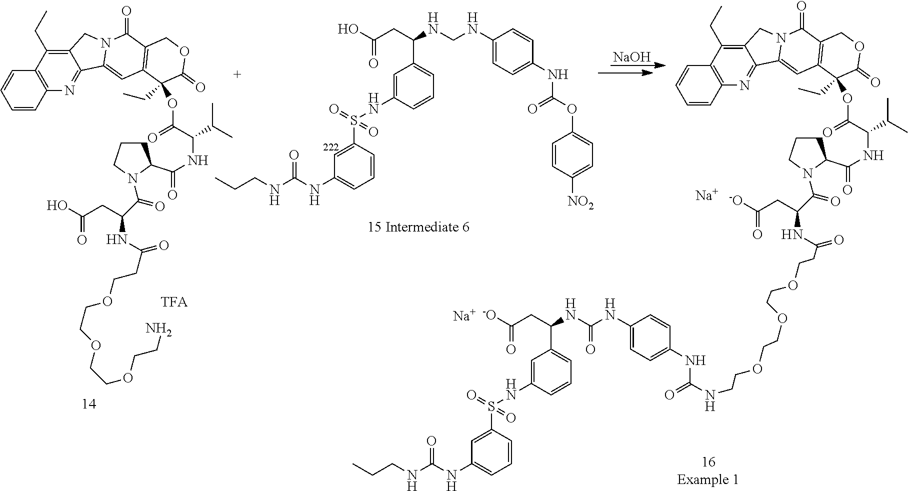

### Method for Treatment:

The present invention also relates to a method for using the compounds and compositions thereof, to treat mammalian hyper-proliferative disorders. This method comprises administering to a mammal in need thereof, including a human, an amount of the compound, which is effective to treat the disorder. Hyper-proliferative disorders include but are not limited to solid tumors, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukemias.

Examples of breast cancer include, but are not limited to invasive ductal carcinoma, invasive lobular carcinoma, ductal carcinoma in situ, and lobular carcinoma in situ.

Examples of cancers of the respiratory tract include, but are not limited to small-cell and non-small-cell lung carcinoma, as well as bronchial adenoma and pleuropulmonary blastoma.

Examples of brain cancers include, but are not limited to brain stem and hypophtalmic glioma, cerebellar and cerebral astrocytoma, medulloblastoma, ependymoma, as well as neuroectodermal and pineal tumor. Tumors of the male reproductive organs include, but are not limited to prostate and testicular cancer. Tumors of the female reproductive organs include, but are not limited to endometrial, cervical, ovarian, vaginal, and vulvar cancer, as well as sarcoma of the uterus.

Tumors of the digestive tract include, but are not limited to anal, colon, colorectal, esophageal, gallbladder, gastric, pancreatic, rectal, small intestine, and salivary gland cancers.

Tumors of the urinary tract include, but are not limited to bladder, penile, kidney, renal pelvis, ureter, and urethral cancers.

Eye cancers include, but are not limited to intraocular melanoma and retinoblastoma.

Examples of liver cancers include, but are not limited to hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to squamous cell carcinoma, Kaposi's sarcoma, malignant melanoma, Merkel cell skin cancer, and non-melanoma skin cancer.

Head-and-neck cancers include, but are not limited to laryngeal/hypopharyngeal/nasopharyngeal/oropharyngeal cancer, and lip and oral cavity cancer.

Lymphomas include, but are not limited to AIDS-related lymphoma, non-Hodgkin's lymphoma, cutaneous T-cell lymphoma, Hodgkin's disease, and lymphoma of the central nervous system.

Sarcomas include, but are not limited to sarcoma of the soft tissue, osteosarcoma, malignant fibrous histiocytoma, lymphosarcoma, and rhabdomyosarcoma.

Leukemias include, but are not limited to acute myeloid leukemia, acute lymphoblastic leukemia, chronic lymphocytic leukemia, chronic myelogenous leukemia, and hairy cell leukemia.

These disorders have been well characterized in humans, but also exist with a similar etiology in other mammals, and can be treated by administering pharmaceutical compositions of the present invention.

Based upon standard laboratory techniques known to evaluate compounds useful for the treatment of hyper-proliferative disorders, by standard toxicity tests and by standard pharmacological assays for the determination of treatment of the conditions identified above in mammals, and by comparison of these results with the results of known medicaments that are used to treat these conditions, the effective dosage of the compounds of this invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered will generally range from about 0.001 mg/kg to about 200 mg/kg body weight per day, and preferably from about 0.01 mg/kg to about 20 mg/kg body weight per day. Clinically useful dosing schedules will range from one to three times a day dosing to once every four weeks dosing. In addition, it is possible for “drug holidays”, in which a patient is not dosed with a drug for a certain period of time, to be beneficial to the overall balance between pharmacological effect and tolerability. It is possible for a unit dosage to contain from about 0.5 mg to about 1500 mg of active ingredient, and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

The present invention further provides the use of the compound of the invention for the preparation of a pharmaceutical compositions for the treatment of the aforesaid disorders.

### Administration

It is possible for the compounds according to the invention to have systemic and/or local activity. For this purpose, they can be administered in a suitable manner, such as, for example, via the oral, parenteral, pulmonary, nasal, sublingual, lingual, buccal, rectal, vaginal, dermal, transdermal, conjunctival, otic route or as an implant or stent.

For these administration routes, it is possible for the compounds according to the invention to be administered in suitable administration forms.

For oral administration, it is possible to formulate the compounds according to the invention to dosage forms known in the art that deliver the compounds of the invention rapidly and/or in a modified manner, such as, for example, tablets (uncoated or coated tablets, for example with enteric or controlled release coatings that dissolve with a delay or are insoluble), orally-disintegrating tablets, films/wafers, films/lyophylisates, capsules (for example hard or soft gelatine capsules), sugar-coated tablets, granules, pellets, powders, emulsions, suspensions, aerosols or solutions. It is possible to incorporate the compounds according to the invention in crystalline and/or amorphised and/or dissolved form into said dosage forms.

Parenteral administration can be effected with avoidance of an absorption step (for example intravenous, intraarterial, intracardial, intraspinal or intralumbal) or with inclusion of absorption (for example intramuscular, subcutaneous, intracutaneous, percutaneous or intraperitoneal). Administration forms which are suitable for parenteral administration are, inter alia, preparations for injection and infusion in the form of solutions, suspensions, emulsions, lyophylisates or sterile powders.

Examples which are suitable for other administration routes are pharmaceutical forms for inhalation [inter alia powder inhalers, nebulizers], nasal drops, nasal solutions, nasal sprays; tablets/films/wafers/capsules for lingual, sublingual or buccal administration; suppositories; eye drops, eye ointments, eye baths, ocular inserts, ear drops, ear sprays, ear powders, ear-rinses, ear tampons; vaginal capsules, aqueous suspensions (lotions, mixturae agitandae), lipophilic suspensions, emulsions, ointments, creams, transdermal therapeutic systems (such as, for example, patches), milk, pastes, foams, dusting powders, implants or stents.

The compounds according to the invention can be incorporated into the stated administration forms. This can be effected in a manner known per se by mixing with pharmaceutically suitable excipients.

Pharmaceutically suitable excipients include, inter alia,
fillers and carriers (for example cellulose, microcrystalline cellulose (such as, for example, Avicel®), lactose, mannitol, starch, calcium phosphate (such as, for example, Di-Cafos®)),
ointment bases (for example petroleum jelly, paraffins, triglycerides, waxes, wool wax, wool wax alcohols, lanolin, hydrophilic ointment, polyethylene glycols),
bases for suppositories (for example polyethylene glycols, cacao butter, hard fat),
solvents (for example water, ethanol, isopropanol, glycerol, propylene glycol, medium chain-length triglycerides fatty oils, liquid polyethylene glycols, paraffins),
surfactants, emulsifiers, dispersants or wetters (for example sodium dodecyl sulfate), lecithin, phospholipids, fatty alcohols (such as, for example, Lanette®), sorbitan fatty acid esters (such as, for example, Span®), polyoxyethylene sorbitan fatty acid esters (such as, for example, Tween®), polyoxyethylene fatty acid glycerides (such as, for example, Cremophor®), polyoxethylene fatty acid esters, polyoxyethylene fatty alcohol ethers, glycerol fatty acid esters, poloxamers (such as, for example, Pluronick),
buffers, acids and bases (for example phosphates, carbonates, citric acid, acetic acid, hydrochloric acid, sodium hydroxide solution, ammonium carbonate, trometamol, triethanolamine),
isotonicity agents (for example glucose, sodium chloride),
adsorbents (for example highly-disperse silicas),
viscosity-increasing agents, gel formers, thickeners and/or binders (for example polyvinylpyrrolidone, methylcellulose, hydroxypropylmethylcellulose, hydroxypropylcellulose, carboxymethylcellulose-sodium, starch, carbomers, polyacrylic acids (such as, for example, Carbopol®); alginates, gelatine),
disintegrants (for example modified starch, carboxymethylcellulose-sodium, sodium starch glycolate (such as, for example, Explotab®), cross-linked polyvinylpyrrolidone, croscarmellose-sodium (such as, for example, AcDiSol®)),
flow regulators, lubricants, glidants and mould release agents (for example magnesium stearate, stearic acid, talc, highly-disperse silicas (such as, for example, Aerosil®)),
coating materials (for example sugar, shellac) and film formers for films or diffusion membranes which dissolve rapidly or in a modified manner (for example polyvinylpyrrolidones (such as, for example, Kollidon®), polyvinyl alcohol, hydroxypropylmethylcellulose, hydroxypropylcellulose, ethylcellulose, hydroxypropylmethylcellulose phthalate, cellulose acetate, cellulose acetate phthalate, polyacrylates, polymethacrylates such as, for example, Eudragit®)),
capsule materials (for example gelatine, hydroxypropylmethylcellulose),
synthetic polymers (for example polylactides, polyglycolides, polyacrylates, polymethacrylates (such as, for example, Eudragit®), polyvinylpyrrolidones (such as, for example, Kollidon®), polyvinyl alcohols, polyvinyl acetates, polyethylene oxides, polyethylene glycols and their copolymers and blockcopolymers),
plasticizers (for example polyethylene glycols, propylene glycol, glycerol, triacetine, triacetyl citrate, dibutyl phthalate),
penetration enhancers,
stabilisers (for example antioxidants such as, for example, ascorbic acid, ascorbyl palmitate, sodium ascorbate, butylhydroxyanisole, butylhydroxytoluene, propyl gallate),
preservatives (for example parabens, sorbic acid, thiomersal, benzalkonium chloride, chlorhexidine acetate, sodium benzoate),
colourants (for example inorganic pigments such as, for example, iron oxides, titanium dioxide),
flavourings, sweeteners, flavour- and/or odour-masking agents.

The present invention furthermore relates to a pharmaceutical composition which comprise at least one compound according to the invention, conventionally together with one or more pharmaceutically suitable excipient(s), and to their use according to the present invention.

### Combinations

In accordance with another aspect, the present invention covers pharmaceutical combinations, in particular medicaments, comprising at least one compound of general formula (I) or (Ta) of the present invention and at least one or more further active ingredients, in particular for the treatment and/or prophylaxis of a hyperproliferative disorder.

The term “combination” in the present invention is used as known to persons skilled in the art, it being possible for said combination to be a fixed combination, a non-fixed combination or a kit-of-parts.

A “fixed combination” in the present invention is used as known to persons skilled in the art and is defined as a combination wherein, for example, a first active ingredient, such as one or more compounds of general formula (I) of the present invention, and a further active ingredient are present together in one unit dosage or in one single entity. One example of a “fixed combination” is a pharmaceutical composition wherein a first active ingredient and a further active ingredient are present in admixture for simultaneous administration, such as in a formulation. Another example of a “fixed combination” is a pharmaceutical combination wherein a first active ingredient and a further active ingredient are present in one unit without being in admixture.

A non-fixed combination or “kit-of-parts” in the present invention is used as known to persons skilled in the art and is defined as a combination wherein a first active ingredient and a further active ingredient are present in more than one unit. One example of a non-fixed combination or kit-of-parts is a combination wherein the first active ingredient and the further active ingredient are present separately. It is possible for the components of the non-fixed combination or kit-of-parts to be administered separately, sequentially, simultaneously, concurrently or chronologically staggered.

The compounds of the present invention can be administered as the sole pharmaceutical agent or in combination with one or more other pharmaceutically active ingredients where the combination causes no unacceptable adverse effects. The present invention also covers such pharmaceutical combinations. For example, the compounds of the present invention can be combined with known active ingredients for the treatment and/or prophylaxis of a hyperproliferative disorder.

Examples of active ingredients for the treatment and/or prophylaxis of a hyperproliferative disorder include:

131I-chTNT, abarelix, abemaciclib, abiraterone, acalabrutinib, aclarubicin, adalimumab, ado-trastuzumab emtansine, afatinib, aflibercept, aldesleukin, alectinib, alemtuzumab, alendronic acid, alitretinoin, altretamine, amifostine, aminoglutethimide, hexyl aminolevulinate, amrubicin, amsacrine, anastrozole, ancestim, anethole dithiolethione, anetumab ravtansine, angiotensin II, antithrombin III, apalutamide, aprepitant, arcitumomab, arglabin, arsenic trioxide, asparaginase, atezolizumab, avelumab, axicabtagene ciloleucel, axitinib, azacitidine, basiliximab, belotecan, bendamustine, besilesomab, belinostat, bevacizumab, bexarotene, bicalutamide, bisantrene, bleomycin, blinatumomab, bortezomib, bosutinib, buserelin, brentuximab vedotin, brigatinib, busulfan, cabazitaxel, cabozantinib, calcitonine, calcium folinate, calcium levofolinate, capecitabine, capromab, carbamazepine carboplatin, carboquone, carfilzomib, carmofur, carmustine, catumaxomab, celecoxib, celmoleukin, ceritinib, cetuximab, chlorambucil, chlormadinone, chlormethine, cidofovir, cinacalcet, cisplatin, cladribine, clodronic acid, clofarabine, cobimetinib, copanlisib, crisantaspase, crizotinib, cyclophosphamide, cyproterone, cytarabine, dacarbazine, dactinomycin, daratumumab, darbepoetin alfa, dabrafenib, dasatinib, daunorubicin, decitabine, degarelix, denileukin diftitox, denosumab, depreotide, deslorelin, dianhydrogalactitol, dexrazoxane, dibrospidium chloride, dianhydrogalactitol, diclofenac, dinutuximab, docetaxel, dolasetron, doxifluridine, doxorubicin, doxorubicin+estrone, dronabinol, durvalumab, eculizumab, edrecolomab, elliptinium acetate, elotuzumab, eltrombopag, enasidenib, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, ethinylestradiol, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim, GM-CSF, goserelin, granisetron, granulocyte colony stimulating factor, histamine dihydrochloride, histrelin, hydroxycarbamide, I-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, inotuzumab ozogamicin, interferon alfa, interferon beta, interferon gamma, iobitridol, iobenguane (123I), iomeprol, ipilimumab, irinotecan, Itraconazole, ixabepilone, ixazomib, lanreotide, lansoprazole, lapatinib, lasocholine, lenalidomide, lenvatinib, lenograstim, lentinan, letrozole, leuprorelin, levamisole, levonorgestrel, levothyroxine sodium, lisuride, lobaplatin, lomustine, lonidamine, lutetium Lu 177 dotatate, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, midostaurin, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphine sulfate, mvasi, nabilone, nabiximols, nafarelin, naloxone+pentazocine, naltrexone, nartograstim, necitumumab, nedaplatin, nelarabine, neratinib, neridronic acid, netupitant/palonosetron, nivolumab, pentetreotide, nilotinib, nilutamide, nimorazole, nimotuzumab, nimustine, nintedanib, niraparib, nitracrine, nivolumab, obinutuzumab, octreotide, ofatumumab, olaparib, olaratumab, omacetaxine mepesuccinate, omeprazole, ondansetron, oprelvekin, orgotein, orilotimod, osimertinib, oxaliplatin, oxycodone, oxymetholone, ozogamicine, p53 gene therapy, paclitaxel, palbociclib, palifermin, palladium-103 seed, palonosetron, pamidronic acid, panitumumab, panobinostat, pantoprazole, pazopanib, pegaspargase, PEG-epoetin beta (methoxy PEG-epoetin beta), pembrolizumab, pegfilgrastim, peginterferon alfa-2b, pembrolizumab, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyestradiol phosphate, polyvinylpyrrolidone+sodium hyaluronate, polysaccharide-K, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifene, raltitrexed, ramosetron, ramucirumab, ranimustine, rasburicase, razoxane, refametinib, regorafenib, ribociclib, risedronic acid, rhenium-186 etidronate, rituximab, rolapitant, romidepsin, romiplostim, romurtide, rucaparib, samarium (153Sm) lexidronam, sargramostim, sarilumab, satumomab, secretin, siltuximab, sipuleucel-T, sizofiran, sobuzoxane, sodium glycididazole, sonidegib, sorafenib, stanozolol, streptozocin, sunitinib, talaporfin, talimogene laherparepvec, tamibarotene, tamoxifen, tapentadol, tasonermin, teceleukin, technetium (99mTc) nofetumomab merpentan, 99mTc-HYNIC-[Tyr3]-octreotide, tegafur, tegafur+gimeracil+oteracil, temoporfin, temozolomide, temsirolimus, teniposide, testosterone, tetrofosmin, thalidomide, thiotepa, thymalfasin, thyrotropin alfa, tioguanine, tisagenlecleucel, tocilizumab, topotecan, toremifene, tositumomab, trabectedin, trametinib, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valatinib, valrubicin, vandetanib, vapreotide, vemurafenib, vinblastine, vincristine, vindesine, vinflunine, vinorelbine, vismodegib, vorinostat, vorozole, yttrium-90 glass microspheres, zinostatin, zinostatin stimalamer, zoledronic acid, zorubicin.

### Abbreviations

The following table lists the abbreviations used herein.
Abu—γ-amino butyric acid
ACN—acetonitrile
Boc—tert.-butyloxycarbonyl
Bzl—Benzyl
DCM—dichloromethane
DIEA—diisopropyl ethyl amine (Hünig's base)
DMAP—dimethylamino pyridine
DMF—dimethyl formamide
DMSO—dimethyl sulphoxide
EDCI—1-Ethyl-3-(3-dimethylaminopropyl)carbodiimid
ee—enantiomeric excess
FCS—fetal calf serum
Fmoc—fluorenyl-9-methoxycarbonyl
HATU—2-(1H-7-Azabenzotriazol-1-yl)-1,1,3,3-tetramethyluronium hexafluorophosphate
HPLC—high-performance liquid chromatography
MTBE—methyl tert.-butyl ether
NMP—N-methyl pyrrolidone,
RP—reverse phase
rt—room temperature
RTV—relative tumor volume
TFA—trifluoroacetic acid
THF—tetrahydrofuran
TLC—thin-layer chromatography

The various aspects of the invention described in this application are illustrated by the following examples which are not meant to limit the invention in any way.

The example testing experiments described herein serve to illustrate the present invention and the invention is not limited to the examples given.

### Experimental Section

All reagents, for which the synthesis is not described in the experimental part, are either commercially available, or are known compounds or may be formed from known compounds by known methods by a person skilled in the art.

The compounds and intermediates produced according to the methods of the invention may require purification. Purification of organic compounds is well known to the person skilled in the art and there may be several ways of purifying the same compound. In some cases, no purification may be necessary. In some cases, the compounds may be purified by crystallization. In some cases, impurities may be stirred out using a suitable solvent. In some cases, the compounds may be purified by chromatography, particularly flash column chromatography, using for example prepacked silica gel cartridges, e.g. Biotage SNAP cartidges KP-Sil® or KP-NH® in combination with a Biotage autopurifier system (SP4® or Isolera Four®) and eluents such as gradients of hexane/ethyl acetate or DCM/methanol. In some cases, the compounds may be purified by preparative HPLC using for example a Waters autopurifier equipped with a diode array detector and/or on-line electrospray ionization mass spectrometer in combination with a suitable prepacked reverse phase column and eluents such as gradients of water and acetonitrile which may contain additives such as trifluoroacetic acid, formic acid or aqueous ammonia.

In some cases, purification methods as described above can provide those compounds of the present invention which possess a sufficiently basic or acidic functionality in the form of a salt, such as, in the case of a compound of the present invention which is sufficiently basic, a trifluoroacetate or formate salt for example, or, in the case of a compound of the present invention which is sufficiently acidic, an ammonium salt for example. A salt of this type can either be transformed into its free base or free acid form, respectively, by various methods known to the person skilled in the art, or be used as salts in subsequent biological assays. It is to be understood that the specific form (e.g. salt, free base etc.) of a compound of the present invention as isolated and as described herein is not necessarily the only form in which said compound can be applied to a biological assay in order to quantify the specific biological activity.

### UPLC-MS Standard Procedures:

Analytical UPLC-MS was performed as described below. The masses (m/z) are reported from the positive mode electrospray ionisation unless the negative mode is indicated (ESI−). In most of the cases method 1 is used. If not, it is indicated.

### HPLC- and LC-MS-Methods:

Method 0:

The mass determinations were carried out by high-performance liquid chromatography-mass spectrometry (HPLC-MS) using the electron spray ionization (ESI) method or by FAB or MALDI mass spectroscopy.

Method 1 (LC-MS):

Instrument: Waters ACQUITY SQD UPLC System; Column: Waters Acquity UPLC HSS T3 1.8μ50×1 mm; Eluent A: 1 l Water+0.25 mL 99% ige formic acid, Eluent B: 1 l acetonitrile+0.25 mL 99% formic acid; Gradient: 0.0 min 90% A→1.2 min 5% A→2.0 min 5% A Stove: 50° C.; Flow: 0.40 mL/min; UV-Detection: 208-400 nm.

### EXAMPLES

### Starting Materials and Intermediates

### Intermediate 1

### (3R)-3-(3-aminophenyl)-3-[(tert-butoxycarbonyl)amino]propanoic Acid

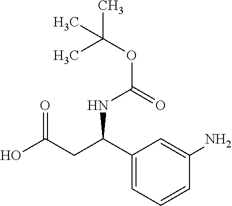 [SMILES: CC(C)(C)OC(=O)N[C@H](CC(=O)O)c1cccc(N)c1]

A mixture of 151 g of 3-nitrobenzaldehyde, 94 g of ammonium acetate, 127 g of malonic acid and 1 L of 2-propanol was heated under reflux for 5 h. The solution was filtered and the precipitate was washed with 0.7 L of hot 2-propanol. The crude product was dried in vacuo, suspended in 1.5 L of water, treated with 1 N hydrochloric acid and filtered. The filtrate was concentrated to yield 146 g.

NMR (400 MHz, D₄-methanol): δ=3.09 (m, 2H), 4.88 (m, 1H), 7.74 (t, 1H), 7.90 (d, 1H), 8.33 (d, 1 H), 8.43 (s, 1H).

20 g (95 mmol) of this intermediate and 31.2 g of di-tert-butyl dicarbonate were dissolved in 150 mL of a dioxane/water mixture (1:1) and 33 mL of DIEA were added. The mixture was stirred for about 90 min until full dissolution is observed. After solvent vaporation the remaining residue was dissolved in 1 L DCM and 3 times extracted with 500 mL of 5% citric acid. The organic phase was concentrated and the product precipitated with a mixture of DCM/diethylether/petrolether 1:1:1 and filtrated. After drying 23.5 g (80%) of the desired product were obtained.

5 g (16.1 mmol) of this intermediate and 3.095 g (23 mmol) (2R)-2-amino-2-phenylethanol were dissolved in acetonitrile and left at 0° C. for 3 days. The precipitate was filtered, dissolved in DCM and 2 times extracted with 5% citric acid. The organic phase was dried upon sodium sulfate and evaporated. This procedure was repeated twice. 1.52 g (30%) of the desired product were obtained with an ee of 95% and an [α]_{D}²⁵=+34.4°/methanol.

1500 mg (0.243 mmol) of this intermediate were dissolved in 100 mL methanol and hydrogenated on palladium/carbon for 30 min under normal pressure. The catalyst was separated off, the solution was concentrated, digested with diethyl ether, filtrated and the residue was dried in vacuo. 1334 mg (98%) of the title compound were obtained.

[DC: (Dichlormethan/Methanol/Ammoniak (17% ig) (15:4:0.5); R_{f}=0.18].

### Intermediate 2

### (3R)-3-[(tert-butoxycarbonyl)amino]-3-{3-[({3-[(propylcarbamoyl)amino]phenyl}sulfonyl)amino]phenyl}propanoic Acid

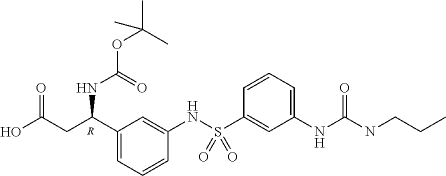

8300 mg (29.6 mmol) of intermediate 1 and 9843 mg (44.4 mmol) of 3-nitrobenzenesulfonyl chloride were dissolved in 400 ml DCM/DMF 1:1 and 7.2 mL pyridine were added. The mixture was stirred overnight at rt. Then the mixture was diluted with 200 mL DCM and extracted 3 times with 50 mL of 5% citric acid. The organic phase was concentrated. After drying the remaining residue 13.8 g (quant.) of (3R)-3-[(tert-butoxycarbonyl)amino]-3-(3-{[(3-nitrophenyl) sulfonyl]amino}phenyl)propanoic acid were obtained.

[DC: (Dichlormethan/Methanol/Ammoniak (17% ig) (15:4:0.5); R_{f}=0.2].

13800 mg (29.65 mmol) of this intermediate were dissolved in 1000 mL methanol and hydrogenated on palladium/carbon for 5 h at normal pressure. The catalyst was separated off, the solution was concentrated, and the residue was washed with diethyl ether twice and then dried in vacuo. 12240 mg (95%) of (3R)-3-(3-{[(3-aminophenyl)sulfonyl]amino}phenyl)-3-[(tert-butoxycarbonyl)amino]propanoic acid were obtained.

12200 mg (28 mmol) of this intermediate were dissolved in 600 mL dioxane and 5722 mg (67 mmol) of 1-isocyanatopropane were added and the mixture was stirred overnight. The solution was concentrated in vacuo and the remaining residue was purified by flash chromatography with a eluent mixture of DCM/methanol/NH4OH (17%) 15/4/0.5. Relevant fractions were collected and concentrated in vacuo. After drying of the residue in vacuo 11220 mg (67%) of the title compound were obtained.

LC-MS (Method 1): Rt=0.9 min; MS (ESIpos): m/z=521 (M+H)⁺.

### Intermediate 3

### (3R)-3-{[(4-aminophenyl)carbamoyl]amino}-3-{3-[({3-[(propyl carbamoyl)amino]phenyl}sulfonyl)amino]phenyl}propanoic Acid

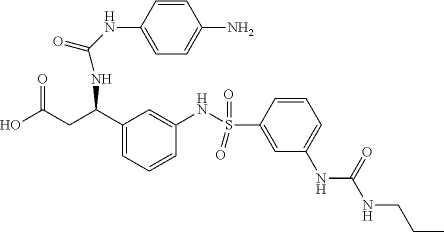 [SMILES: CCCNC(=O)Nc1cccc(S(=O)(=O)Nc2cccc([C@@H](CC(=O)O)NC(=O)Nc3ccc(N)cc3)c2)c1]

400 mg (0.768 mmol) of intermediate 2 were dissolved in 10 mL DCM and 2 mL of trifluoro acetic acid were added. After stirring for 90 min at rt the reaction mixture was concentrated in vacuo. The residue was treated with a 5% solution of disodium carbonate and subsequently dissolved in a mixture of DCM/methanol. After precipitation with diethyl ether, filtration and drying in vacuo 260 mg (81%) of (3R)-3-amino-3-{3-[({3-[(propylcarbamoyl)amino]phenyl}sulfonyl)amino]phenyl}propanoic acid were obtained.

LC-MS (Method 0): Rt=4.11 min; MS: m/z=421=(M+H)⁺

250 mg (0.595 mmol) of this intermediate were dissolved in 15 mL DMF and 117 mg (0.713 mmol) of 1-isocyanato-4-nitrobenzene were added and the solution was stirred for 30 min at rt. Another 30 mg of 1-isocyanato-4-nitrobenzene were added and stirring was continued for 30 min. The solution was concentrated in vacuo and the remaining residue was purified by flash chromatography. After concentration of the relevant fractions in vacuo 160 mg (46%) of (3R)-3-{[(4-nitrophenyl)carbamoyl]amino}-3-{3-[({3-[(propyl carbamoyl)amino]phenyl}sulfonyl)amino]phenyl}propanoic acid were obtained.

LC-MS (Method 0): Rt=5.61 min; MS: m/z=585=(M+H)+142 mg (0.243 mmol) of this intermediate were dissolved in 20 mL methanol/DCM 10:1 and hydrogenated on palladium/carbon for 30 min under normal pressure. The catalyst was separated off, the solution was concentrated, digested with diethyl ether, filtrated and the residue was dried in vacuo. 103 mg (76%) of the title compound were obtained.

LC-MS (Method 0): Rt=4.31 min; MS: m/z=555=(M+H)⁺

¹H-NMR (500 MHz, D₄-methanol): 6=0.93 (t, 3H), 1.5 (m, 2H), 2.74 (d, 2H), 3.1 (dt, 2H), 5.15 (t, 1 H), 6.68 (d, 2H), 6.85 (d, 1H), 7.05 (d, 1H), 7.1 (d, 1H), 7.13 (t, 1H), 7.28-7.4 (m, 3H), 7.6 (s, 1H), 7.66 (d, 1H).

### Intermediate 4

### (4S)-4,11-diethyl-3,14-dioxo-3,4,12,14-tetrahydro-1H-pyrano[3′,4′:6,7]indolizino[1,2-b]quinolin-4-yl L-valinate Trifluoroacetate (1:1)

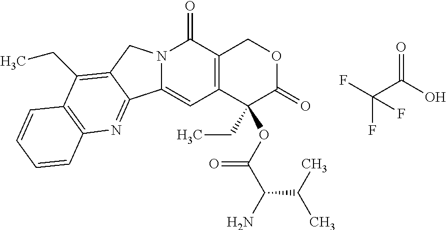

2.59 g (10.6 mmol) of N-(tert-butoxycarbonyl)-valine-N-carboxyanhydride and 0.5 g of 4-(N,N-dimethylamino)-pyridine were added to a stirred suspension of 2 g (5.3 mmol) of (4S)-4,11-diethyl-4-hydroxy-1H-pyrano[3′,4′:6,7]indolizino[1,2-b]quinoline-3,14(4H,12H)-dione (7 ethyl camptothecin, synthesized as described by S. Sawada et al. in Chem. Phar. Bull 1991-39(6)-1445) in 150 ml of absolute dichloromethane. The mixture was stirred at rt for 20 h and subsequently concentrated in vacuo. 8 ml ACN were added to the residue and subsequently 5 mL diethyl ether. The mixture was filtrated and the remaining residue was dried in vacuo. 2964 mg (92%) of the protected intermediate were obtained.

LC-MS (Method 1): Rt=1.19 min; MS (ESIpos): m/z=576 (M+H)+. 2964 mg (5.15 mmol) of this Boc-protected intermediate compound in 6 ml of dichloromethane and 60 ml of anhydrous trifluoroacetic acid was stirred for 30 min. at rt and subsequently sonicated for 1 h. After concentrating in vacuo the product was lyophilized from a mixture of acetonitrile/water. 3.622 g (quant) of the title compound were obtained.

LC-MS (Method 1): Rt=0.68 min; MS (ESIpos): m/z=476 (M+H)⁺.

### Intermediate 5

### (2S)-1-[(19S)-19-(2-tert-butoxy-2-oxoethyl)-2,2-dimethyl-4,17,20-trioxo-3,8,11,14-tetraoxa-5,18-diazaicosan-20-yl]pyrrolidine-2-carboxylic Acid

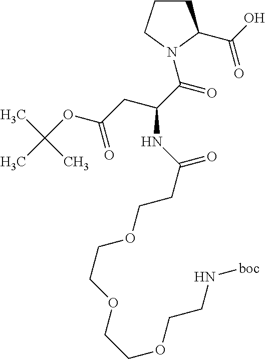

This intermediate 5 was synthesized following classical methods known in peptide chemistry starting with the coupling of 4-tert-butyl 1-(2,5-dioxopyrrolidin-1-yl)N-(tert-butoxycarbonyl)-L-aspartate with benzyl L-prolinate hydrochloride (1:1) in DMF in the presence of DIEA and subsequent cleavage of the benzylester by hydrogenation over palladium/carbon. Subsequently, the tert.-butoxycarbonyl protecting group was removed by stirring a solution of (2S)-1-{(2S)-4-tert-butoxy-2-[(tert-butoxycarbonyl)amino]-4-oxobutanoyl}pyrrolidine-2-carboxylic acid for 15 minutes in a mixture of 15 mL TFA and 100 mL DCM followed by purification via flash chromatography using DCM/methanol 3:1 as eluent. This intermediate was dissolved in DMF and coupled in the presence of DIEA with tert-butyl {2-[2-(2-{3-[(2,5-dioxopyrrolidin-1-yl)oxy]-3-oxopropoxy}ethoxy)ethoxy]ethyl}carbamate (previously obtained by transformation of 2,2-dimethyl-4-oxo-3,8,11,14-tetraoxa-5-azaheptadecan-17-oic acid to the activated ester in DMF with 1-hydroxy pyrrolidine-2,5-dione and EDCI).

LC-MS (Method 1): Rt=0.86 min; MS (ESIpos): m/z=590 (M+H)⁺.

### Intermediate 6

### (3R)-3-{[(4-{[(4-nitrophenoxy)carbonyl]amino}phenyl)carbamoyl]amino}-3-{3-[({3-[(propylcarbamoyl)amino]phenyl}sulfonyl)amino]phenyl}propanoic Acid

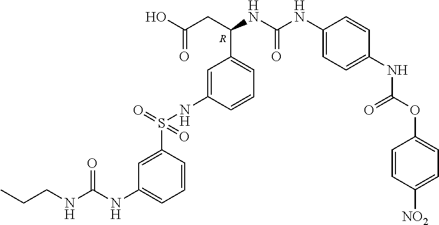

8.99 g (43.3 mmol) 4-nitrophenyl carbonochloridate were dissolved in 1300 mL THF and 12 g (21.64 mmol) of (3R)-3-{[(4-aminophenyl)carbamoyl]amino}-3-{3-[({3-[(propyl carbamoyl)amino]phenyl}sulfonyl)amino]phenyl}propanoic acid were added. The mixture was heated and stirred for 45 min under reflux, and subsequently cooled down to rt and filtrated. The filtrate was concentrated under reduced pressure to a volume of 100 mL. This solution was poured into diethyl ether and the precipitate was filtrated. After drying overnight in vacuo 11.6 g of the title compound were obtained.

LC-MS (Method 1): Rt=0.97 min; MS (ESIpos): m/z=720 (M+H)⁺.

### Intermediate 7: Reference Compound to Integrin Ligand (S-Epimer of Intermediate 3)

### (3S)-3-{[(4-aminophenyl)carbamoyl]amino}-3-{3-[({3-[(propyl carbamoyl)amino]phenyl}sulfonyl) amino]phenyl}propanoic Acid

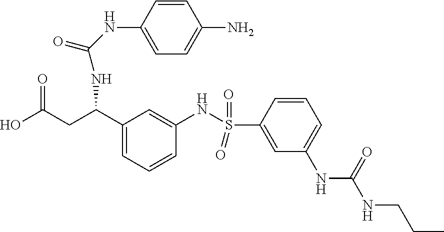 [SMILES: CCCNC(=O)Nc1cccc(S(=O)(=O)Nc2cccc([C@H](CC(=O)O)NC(=O)Nc3ccc(N)cc3)c2)c1]

This compound was synthesized in analogy to the intermediate 3 mentioned above utilizing the epimer of intermediate 1 which was found in the mother liquor during the optical resolution step.

### Example 1: αᵥβ₃ Integrin Conjugate

disodium (4S)-4,11-diethyl-3,14-dioxo-3,4,12,14-tetrahydro-1H-pyrano[3′,4′:6,7]indolizino [1,2-b]quinolin-4-yl 1-{(2S)-2-(carboxylatomethyl)-17-[4-({[(1R)-2-carboxylato-1-{3-[({3-[(propyl-carbamoyl)amino]phenyl}sulfonyl)amino]phenyl}ethyl]carbamoyl}amino)anilino]-4,17-dioxo-7,10,13-trioxa-3,16-diazaheptadecan-1-oyl}-L-prolyl-L-valinate

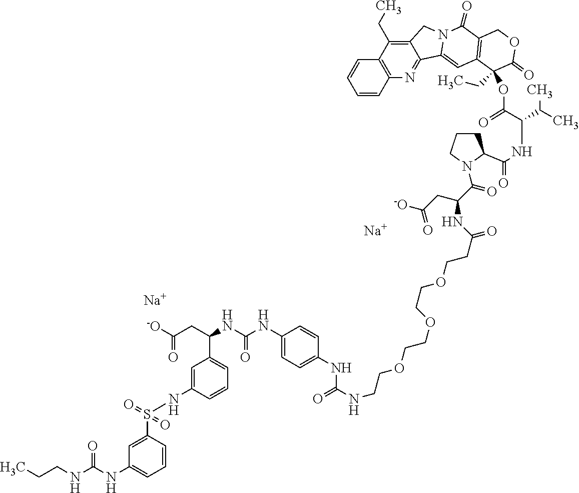

40 mg (68 μmol) of intermediate 4 and 48 mg (81 μmol) of intermediate 5 were dissolved in 6.4 mL DMF and 33.5 mg (88 μmol) HATU and 35 μL DIEA were added. The mixture was stirred at rt for 30 min. The mixture was evaporated and the remaining residue was purified by HPLC. 28 mg (39%) of the protected intermediate were obtained.

LC-MS (Method 1): Rt=1.15 min; MS (ESIpos): m/z=1047 (M+H)⁺.

28 mg of this intermediate were dissolved in 2 ml of dichloromethane. 2 ml of anhydrous trifluoroacetic acid were added and the mixture was stirred for 30 min at rt and subsequently sonicated for 1 h. After concentrating in vacuo the product was lyophilized from a mixture of acetonitrile/water. 30 mg (quant.) of the deprotected intermediate were obtained as an orange solid.

LC-MS (Method 1): Rt=0.72 min; MS (ESIpos): m/z=891 (M+H)⁺.

1900 mg (1.89 mmol) of this intermediate were dissolved in 60 mL DMF and 1361 mg (1.89 mmol) of intermediate 6 were added and the mixture was stirred for 2 h at rt. The solution was concentrated in vacuo and the remaining residue was treated with water and 5% citric acid and filtrated. The remaining residue was dissolved in DCM/methanol and diethyl ether was added. The precipitate was filtrated and purified by flash-chromatography with an eluent mixture of DCM/methanol/NH40H (17%) 15/2/0.2->15/4/0.4. Relevant fractions were collected and concentrated in vacuo. After drying of the residue in vacuo 942 mg (34%) of the title compound were obtained.

LC-MS (Method 1): Rt=0.97 min; MS (ESIpos): m/z=1471 (M+H)⁺.

20 mg (14 μmol) of this intermediate were dissolved in 4 mL dioxane/water 1:1 and 30 μL (30 μmol) of a 1 n aqueous solution of sodium hydroxide were added and the mixture was sonicated for 5 min at rt and lyophilized. 21 mg (quant) of the title compound were obtained.

LC-MS (Method 1): Rt=0.97 min; MS (ESIpos): m/z=1471 (M−2Na⁺+2H⁺+H)⁺.

### Example 2: Reference Compound of Example 1 (S-Epimer)

### disodium (4S)-4,11-diethyl-3,14-dioxo-3,4,12,14-tetrahydro-1H-pyrano[3′,4′:6,7]indolizino [1,2-b]quinolin-4-yl 1-{(2S)-2-(carboxylatomethyl)-17-[4-({[(1S)-2-carboxylato-1-{3-[({3-[(propyl-carbamoyl)amino]phenyl}sulfonyl)amino]phenyl}ethyl]carbamoyl}amino)anilino]-4,17-dioxo-7,10,13-trioxa-3,16-diazaheptadecan-1-oyl}-L-prolyl-L-valinate

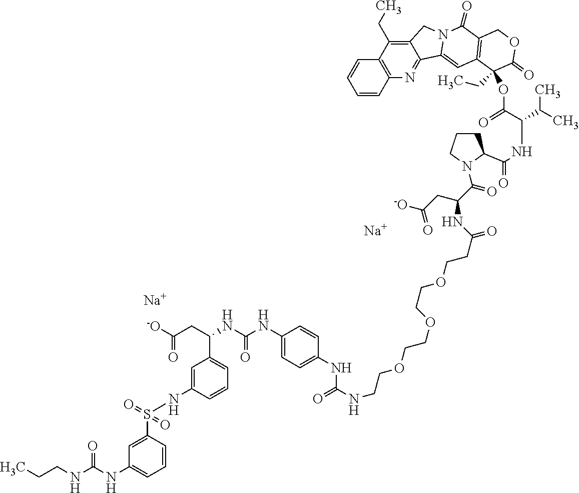

This compound was synthesized in analogy to example 1 utilizing the epimer of the αᵥβ₃ ligand of intermediate 7.

### Biological Evaluation of the Preferred Toxophore 7-Ethyl Camptothecin and the Conjugate of Example 1

### In Vitro Tests for Determining Cellular Permeability

### Caco-2:

The cell permeability of a substance can be investigated by means of in vitro testing in a flux assay using Caco-2 cells [M. D. Troutman and D. R. Thakker, Pharm. Res. 20 (8), 1210-1224 (2003)]. For this purpose, the cells were cultured for 15-16 days on 24-well filter plates. For the determination of permeation, the respective test substance was applied in a HEPES buffer to the cells either apically (A) or basally (B) and incubated for 2 hours. After 0 hours and after 2 hours, samples were taken from the cis and trans compartments. The samples were separated by HPLC (Agilent 1200, Böblingen, Germany) using reverse phase columns. The HPLC system was coupled via a Turbo Ion Spray Interface to a Triple Quadropol mass spectrometer API 4000 (AB SCIEX Deutschland GmbH, Darmstadt, Germany). The permeability was evaluated on the basis of a Papp value, which was calculated using the formula published by Schwab et al. [D. Schwab et al., J. Med. Chem. 46, 1716-1725 (2003)]. A substance was classified as actively transported when the ratio of Papp (B-A) to Papp (A-B) (efflux ratio) was >2 or <0.5.

In this assay the toxophore (4S)-4,11-diethyl-4-hydroxy-1H-pyrano[3′,4′:6,7]indolizino[1,2-b]quinoline-3,14(4H,12H)-dione (7-Ethyl-camptothecin), which was employed in the conjugate of example 1 shows a very good permeability of Pₐₚₚ A->B=171 nm/s and a low efflux ratio of 1. This favourably compares to the profile of SN38, the toxophore released from Irinotecan which shows a significantly lower permeability of Pₐₚₚ A->B=8 nm/s and an efflux ratio of 36. New data for SN38: Permeability of Papp A->B=20 nm/s and an efflux ratio of 9.

P-glycoprotein (p-GP) Assay:

Many tumor cells express transporter proteins for drugs, and this frequently accompanies the development of resistance towards cytostatics. Substances which are not substrates of such transporter proteins, such as P-glycoprotein (P-gp) or BCRP, for example, could therefore exhibit an improved activity profile.

The substrate properties of a substance for P-gp (ABCB1) were determined by means of a flux assay using LLC-PK1 cells which overexpress P-gp (L-MDR1 cells) [A. H. Schinkel et al., J. Clin. Invest. 96, 1698-1705 (1995)]. For this purpose, the LLC-PK1 cells or L-MDR1 cells were cultured on 96-well filter plates for 3-4 days. For determination of the permeation, the respective test substance, alone or in the presence of an inhibitor (such as ivermectin or verapamil, for example), was applied in a HEPES buffer to the cells either apically (A) or basally (B) and incubated for 2 hours. After 0 hours and after 2 hours, samples were taken from the cis and trans compartments. The samples were separated by HPLC using reverse phase columns. The HPLC system was coupled via a Turbo Ion Spray Interface to a Triple Quadropol mass spectrometer API 3000 (Applied Biosystems Applera, Darmstadt, Germany). The permeability was evaluated on the basis of a Papp value, which was calculated using the formula published by Schwab et al. [D. Schwab et al., J. Med. Chem. 46, 1716-1725 (2003)]. A substance was classified as P-gp substrate when the efflux ratio of Papp (B-A) to Papp (A-B) was >2.

In this assay the toxophore (4S)-4,11-diethyl-4-hydroxy-1H-pyrano[3′,4′:6,7]indolizino[1,2-b]quinoline-3,14(4H,12H)-dione (7-Ethyl-camptothecin), which was employed in the conjugate of example 1 shows a very good permeability of Pₐₚₚ A->B=196 nm/s and a low efflux ratio of 0.6. This favourably compares to the profile of SN38, the toxophore released from Irinotecan which shows a significantly lower permeability of Pₐₚₚ A->B=10 nm/s and an efflux ratio of 16.

### Cytotoxicicity In Vitro Against NCI-H1975 and its Transporter Mutants

The cytotoxic activity of 7-Ethyl camptothecin is not negatively affected when tumor cells NCI-H1975 was transfected with drug transporters p-Glycoprotein (P-gp) and breast cancer resistant protein (BCRP) which is in strong contrast to SN38.

TABLE 1
Cytotoxicicity in vitro against NCI-H1975 and
its transporter mutants

| | NCI- | NCI- | NCI- |
|---|---|---|---|
| | H1975 | H1975-P-gp | H1975-BCRP |
| | IC₅₀ | IC₅₀ | IC₅₀ |
| Compound | [nM] | [nM] | [nM] |
| 7Et-CPT | 19 | 34 | 27 |
| SN38 | 45 | 141 | 512 |

αᵥβ₃ Binding Test αᵥβ₃ from human A375 cells was purified analogously to a procedure described by Wong et al. in Molecular Pharmacology 50, 529-537 (1996). In each case, 10 μL of αᵥβ₃ (5 ng) in TBS pH 7.6, 2 mM CaCl₂, 1 mM MgCl₂, 1% n-octylglucopyranoside (Sigma); 10 μL of test substance in TBS pH 7.6, 0.1% DMSO and 45 μL of TBS pH 7.6, 2 mM CaCl₂, 1 mM MgCl₂, 1 mM MnCl₂ were incubated at room temperature for 1 h. In each case, 25 μL of WGA SPA beads (Amersham, 4 mg/ml) and 10 μL of echistatin (0.1 μCi, Amersham, chloramine-T labelled) were then added. After 16 h at room temperature, the samples were measured in a scintillation measuring apparatus (Wallac 1450). The test results are shown in Table 2 below.

TABLE 2
IC₅₀ values of the binding to the αᵥβ₃ receptor

| Example | IC₅₀ [nM] |
|---|---|
| 1 | 29 |
| 2 | 700 |

### Elastase Cleavability

### Cytotoxicity In Vitro in the Presence and Absence of Elastase

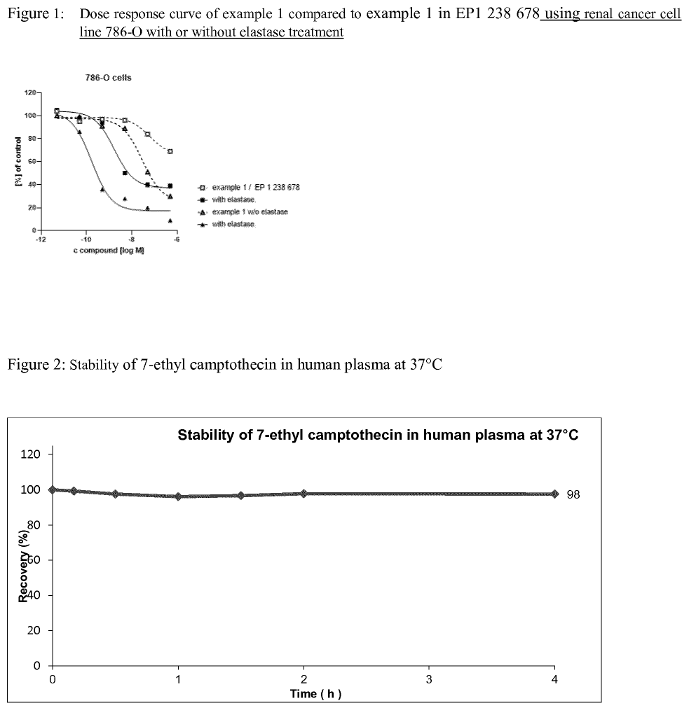

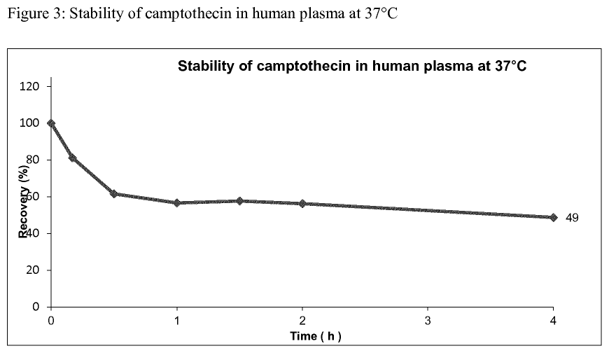

Cultivation of cells was performed according to standard procedures with the media recommended by the provider. The cells in a total volume of 100 μL were seeded in a 96-well plate with white bottom (#3610). After a 24 h incubation period at 37° C. and 5% CO₂, the medium was changed by adding 90 μL fresh medium. The treatment starts by adding the test compound to the cells in 10 μl of culture medium. Concentrations from 10⁻⁵ M to 10⁻¹³ M in triplicates were chosen followed by an incubation at 37° C. and 5% carbon dioxide. One set of samples were only treated with the test compound whereas to an otherwise identically treated second set of samples also 10 nM elastase was pipetted. After 72 h, the proliferation is detected using the MTT assay (ATCC). At the end of the incubation period the MTT reagent is added to all samples for 4 h, followed by lysis of the cells overnight by addition of the detergent. The dye formed was detected at 570 nm. The proliferation of cells which were not treated with test substance but were otherwise identically treated was defined as the 100% value. The dose response curve allows the determination of the respective IC₅₀ values, which are summarized in table 3. (FIG. 1 and table 4).

TABLE 3
IC₅₀ values of example 1 and 2 with and
w/o elastase presence are summarized

| | 786-O cell line | | HT29 cell line | |
|---|---|---|---|---|
| | IC50 [nM] | | IC50 [nM] | |
| Example | w/o elastase | w elastase | w/o elastase | w elastase |
| 1 | 188 | 1.1 | 245 | 8.7 |
| 2 | 268 | 0.17 | >500 | 32 |

TABLE 4
IC₅₀ values of example 1 and example 1 in EP 1 238 678 with and
w/o elastase in a side by side comparison
(elastase with higher enzymatic activity used)

| | 786-O cell line IC50 [nM] | | |
|---|---|---|---|
| Example | w/o elastase | w elastase | Specificity factor |
| 1 | 28 | 0.17 | 165 |
| 1/EP 1 238 678 | 63 | 1.7 | 37 |

The presence of neutrophil elastase elicits a significant improvement of the cytotoxicity of the compound using the renal cancer cell line 786-O. The compounds also reveal a pronounced dependency on elastase using the colon cancer cell line HT29. Again elastase induced cleavage evokes a dramatic increase of the cytotoxic effect of the compound.

### Solubility of Conjugate in Example 1 as Compared to Conjugate of Example 1 in EP 1 238 678:

Method: For each vehicle to be tested, 0.5-1.0 mg test compound were weighed into a 2 ml Eppendorf vial. 2-3 Glas perls (Ø3 mm) and 1.0 ml vehicle were added. The vial was shaken at 1400 rpm for 24 hrs at room temperature (25° C.). After this time period the supernatant (approx. 230 μl was transferred to a centrifuge tube. After 30 min at 42 000 rpm the solute was transferred to another vial and diluted with DMSO (1:5 and 1:50). These two dilutions were analyzed by HPLC (read out:area)

### HPLC-Method:

Eluent A: 1 ml Trifluoro acetic acid/L water

Eluent B: 1 ml Trifluoro acetic acid/L acetonitril

### Gradient:

| Time [min] | A [%] | B [%] | Flow: [ml/min] |
|---|---|---|---|
| 0.0 | 98 | 2 | 1.5 |
| 0.2 | 98 | 2 | 1.5 |
| 3.3 | 10 | 90 | 1.5 |
| 4.0 | 10 | 90 | 1.5 |
| 4.1 | 98 | 2 | 2.5 |
| 4.7 | 98 | 2 | 2.5 |
| 5.0 | 98 | 2 | 1.5 |

### Column: ZORBAX Extend-C18, 3.0×50 mm, 3.5 μm

Oven temperature: 30° C.

### Detection: 214 and 254 nm

Injection volume: 20 μl

For quantification a calibration curve was obtained from DMSO solution of the test compound (100 μl/ml, 20 μg/ml and 2.5 μg/ml) by employing the same HPLC method.

TABLE 5
Solubility of example 1 and example 1 from EP 1 238 678

| | | 0.9% NaCl in |
|---|---|---|
| Compound | 5% D-Mannitol | H₂O |
| Example 1 | >500 mg/mL | 465 |
| Example 1 from EP 1 238 678 | 200 mg/mL | 200 |

### Stability in Citric Acid Buffer at pH 4 of Conjugate in Example 1 as Compared to Conjugate of Example 1 in EP 1 238 678:

Method: 0.15 mg of the test compound were solved in 0.1 ml dimethylsulfoxide and 0.4 ml acetonitrile. For complete dissolution the HPLC vial with the sample solution was shaken and sonicated. Then 1.0 ml of the respective buffer solution (Citrate buffer pH 4; citric acid/sodium hydroxide/sodium chloride Fluka 33643) was added and the sample was vortexed. The sample solution was analysed by HPLC to determine the amount of the test compound and up to two byproducts at a particular time (0, 1, 2 4, 24 hrs) over a period of 24 h at 37° C. t(0) values resulted from a sample immediately taken after vortexing with buffer at RT. The peak areas (in percentage) were used for quantification.

LC & LC/MS purity analysis: The starting material was analyzed for purity by LC; the 24 h sample was additionally analyzed by LC/MS (Waters Quattro Micro).

| HPLC conditions | Agilent |
|---|---|
| DAD | G4212B |
| Column oven | G1316C |
| Thermostat | G1330B |
| Autosampler | G1367E |
| binary pump | G1312B |

| | A = 1 ml formic acid/L Water B = 1 ml formic acid/L | | | Time | | | Flow |
|---|---|---|---|---|---|---|---|
| Eluent: | ACN | | Gradient: | (min) | A (%) | B (%) | (ml/min) |
| Column: | Nucleodur 100 C18ec 3 μm 50 * 2 mm | | | 0.0 | 98 | 2 | 0.75 |
| Temp.: | 37 | ° C. | | 1.0 | 98 | 2 | 0.75 |
| Detect.: | 214 | nm | | 15.0 | 5 | 95 | 0.75 |
| Flow: | 0.75 | ml/min | | 17.5 | 5 | 95 | 0.75 |
| Inj.: | 8 | μl | | 17.7 | 98 | 2 | 1.50 |
| | | | | 18.2 | 98 | 2 | 1.50 |
| | | | | 18.5 | 98 | 2 | 1.00 |
| | | | | 19.0 | 98 | 2 | 0.75 |

TABLE 6
Stability of example 1 and example 1 from EP 1238 678 in
citric acid buffer at pH 4

| Compound | 4h | 24h |
|---|---|---|
| Example 1 | 100% | 95% |
| Example 1 from EP 1 238 678 | 100% | 74% |

### Plasma Stability of Conjugate in Example 1

### Measurement of Release of Parent Compound in Rat Plasma:

1 mg of the test compound of example 1 was dissolved in a mixture of 1.5 mL dimethylsulfoxide and 1 ml water. For complete dissolution the HPLC vial was shaken and treated with ultrasound. 500 μl of this solution were added to 0.5 mL of rat plasma with vortexing at a temperature of 37° C. Aliquots (10 μL each) were taken at respective time points and analyzed by HPLC to determine the amount of the test compound. All data is given as percent area of the initial compound at t0.

Compound of example 1 is stable in rat plasma for >24 hours.

### Stability of 7-Ethyl Camptothecin (Toxophore of Example 1) and Camptothecin (Toxophore of Example 1 in EP 1 238 678) in Human Plasma:

1 mg of the test compound was solved in 0.5 ml acetonitrile/dimethylsulfoxide 1:1. For complete dissolution the HPLC vial was shaken and sonicated. While vortexing 20 μl of this solution were added to 1 ml 37° C. warm plasma. After 0.17, 0.5, 1, 1.5, 2 and 4 hours the enzymatic reaction was stopped by adding 100 μl of the compound plasma solution to a vial containing 300 μl acetonitrile/buffer pH3 (80:20) at RT. The mixture was centrifuged at 5000 rpm for 10 minutes. The supernatant was analyzed by HPLC to determine the amount of the test compound and up to two byproducts. t(0) values result from a processed sample immediately taken after vortexing with plasma at RT. The peak areas (in percentage) were used for quantification. was

Under the assay conditions 7-Ethyl camptothecin is stable for at least 4 hours whereas in the same time camptothecin is degraded to an extent of about 50%.

### Pharmacokinetics

4 mg of the conjugate of example 1 were dissolved in saline and administered iv to female 786-0 tumor bearing NMRI nu/nu mice. Tumor and plasma samples were collected at different time points and the levels of intact conjugate and of the toxophore 7-ethyl-camptothecin cleaved from the conjugate were determined.

For comparison, 1 mg/kg of 7-ethyl camptothecin was dissolved in a mixture of 5% aqueous dextrose/solutol/DMSO 85/10/5 and administered iv to female, 786-0 tumor bearing NMRI nu/nu mice. Again tumor and plasma samples were collected at different time points and the levels of 7-ethyl-camptothecin were determined.

Finally, for comparison 4 mg of the epimeric reference conjugate of example 23 (with weak αᵥβ₃ binding affinity) were dissolved in saline and administered iv to female 786-0 tumor bearing NMRI nu/nu mice. Tumor and plasma samples were collected at different time points and the levels of intact conjugate and of the toxophore 7-ethyl-camptothecin cleaved from the conjugate were determined.

In the table 4 tumor/plasma ratios of 7-ethyl camptothecin detected in each of these experiments are summarized. Enhanced delivery of 7-ethyl camptothecin to the tumor via the αᵥβ₃ integrin conjugate is demonstrated in comparison to direct administration of the toxophore and to administration of an weakly binding epimeric control conjugate.

TABLE 4

| | Tumor/Plasma | Tumor/Plasma |
|---|---|---|
| Compound | ratio of | enrichment factor |
| administered | 7-ethyl camptothecin | (ratio/ratio) |
| Example 1 | 6.5 | 10.8 |
| Example 2 (reference) | 1.2 | 2 |
| 7-ethyl camptothecin | 0.6 | 1 |

### In Vivo Xenotransplantation Studies

The anti-tumor activities of example 1 was examined in murine xenotransplantation models of human cancer. For this purpose, immunocompromised mice were implanted subcutaneously with tumor cells or tumor fragments. At a mean tumor size of 20-40 mm² animals were randomized into treatment and control groups (n=8 animals/group) and treatment started with vehicle only or example 1 (formulation: phosphate buffered saline (“PBS”); application route: intravenously into the tail vein (“i.v.”)). Intravenous treatments were performed on three consecutive days once daily followed by four days drug holiday without treatments. The tumor size and the body weight were determined at least weekly. The tumor area was detected by means of an electronic caliper [length (mm) x width (mm)]. The experimental groups were ended when the group reached the pre-determined ethical endpoint based on German and European animal welfare regulations. In vivo anti-tumor efficacy is presented as T/C ratio of mean tumor area measured for treatment and control group on the last day at which the vehicle control remained in study (Treatment/Control; mean tumor area of treatment group/mean tumor area of control group. A compound having a T/C below 0.5 is defined as active (i.e., effective). Statistical analysis was assessed using SigmaStat software. A one-way analysis of variance was performed and differences to the control were compared by a pair-wise comparison procedure (Dunn's method).

### Results:

Example 1 showed potent anti-tumor efficacy in different xenograft models of human tumors upon monotherapy treatment. Specifically, example 1 was effective in reduction of tumor area in models of breast, colon, lung, and renal cancer.

TABLE 5
Anti-tumor activity of example 1 in different human
cancer xenograft models in mice.

| Xenograft | Cell line isolated | | | |
|---|---|---|---|---|
| Model | from patient with | Compound | Dose and schedule | T/C |
| MX1 | Breast cancer | Example 1 | 36 mg/kg3 days on, | 0.03* |
| | | | 4 days off, 3 cycles | |
| SW-480 | Colon cancer | Example 1 | 36 mg/kg3 days on, | 0.1* |
| | | | 4 days off, 3 cycles | |
| NCI-H69 | Lung cancer | Example 1 | 40 mg/kg3 days on, | 0.06* |
| | | | 4 days off, 3 cycles | |
| 786-O | Renal cancer | Example 1 | 36 mg/kg3 days on, | 0.19* |
| | | | 4 days off, 3 cycles | |

*P <0.05 (compared to vehicle treated control)
T/C = ratio of the mean tumor area of treatment versus mean tumor area of control group at the last day
at which control group remained within the study

What is claimed is:

1. Compound of the formula (I)
CT-LI—SP-IA (I)
in which
CT is a mono valent radical from the group of a cytotoxic radical, a radical of a cytostatic and a radical of a cytostatic derivative, which can each additionally carry hydroxyl, carboxyl or amino group
LI is a bivalent peptide radical of the formula: -L-Val-L-Pro-L-Asp-
SP is a group of the formula: —C═O—(CH₂)ₓ—O—(CH₂—CH₂—O)_{y}—CH₂—CH₂—(NH)_{z}—C═O— with x=1-5, y=0-15 and z=0-1
IA is a monovalent radical addressing an αᵥβ₃ integrin receptor
and the salts, solvates and solvates of the salts thereof.

2. Compound of the general formula (Ta) 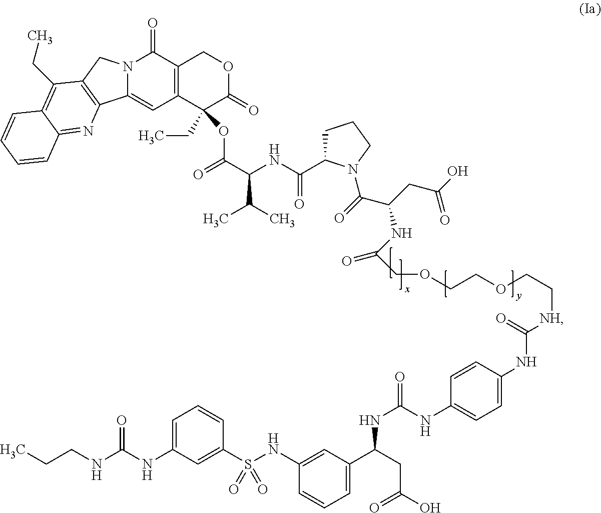
in which x is 1-5 and y=0-15,
and the salts, solvates and solvates of the salts thereof.

3. A compound of any one of claim of claims 1 to 2 wherein x=1-4, and y=0-10.

4. A compound of any one of claim of claims 1 to 2 wherein x=1-2, and y=0-5.

5. Compound of the formula (II) 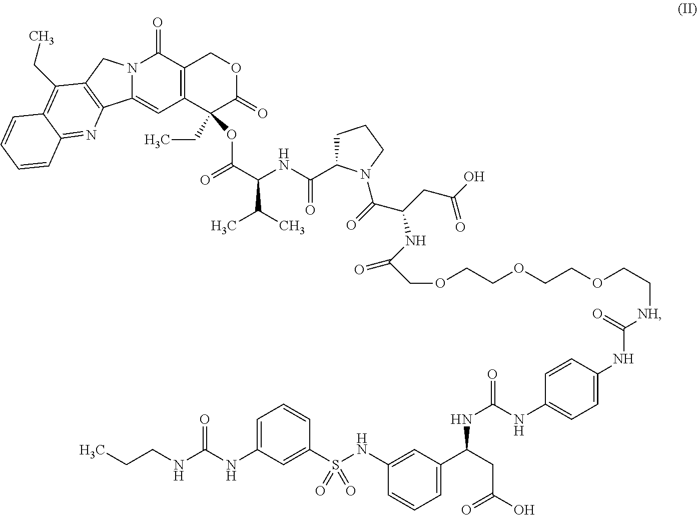 [SMILES: CCCNC(=O)Nc1cccc(S(=O)(=O)Nc2cccc([C@H](CC(=O)O)NC(=O)Nc3ccc(NC(=O)NCCOCCOCCOCC(=O)N[C@@H](CC(=O)O)C(=O)N4CCC[C@H]4C(=O)N[C@H](C(=O)O[C@]4(CC)C(=O)OCc5c4cc4n(c5=O)Cc5c-4nc4ccccc4c5CC)C(C)C)cc3)c2)c1]
and the salts, solvates and solvates of the salts thereof.

6. The compound of claim 5 in form of its disodium salt.

7. Compound as defined in any one of claims 1 to 6 for treatment and/or prevention of diseases.

8. Compound as defined in any one of claims 1 to 6 for treatment and/or prevention of hyperproliferative disorders.

9. Use of a compound as defined in any one of claims 1 to 6 for production of a medicament for treatment and/or prevention of hyperproliferative disorders.

10. Medicament comprising a compound as defined in any one of claims 1 to 6 in combination with one or more inert, nontoxic, pharmaceutically suitable excipients.

11. Medicament of claim 10 for treatment and/or prevention of hyperproliferative disorders.

12. Method for treatment and/or prevention of ophthalmological disorders and cancers or tumors in humans and animals by administering an effective amount of at least one compound as defined in any one of claims 1 to 6, or of a medicament as defined in any one of claims 10 to 11.
